(19) United States
(12) Patent Application Publication
(10) Pub. No.: US 2006/0155110 A1
(43) Pub. Date: Jul. 13, 2006

(54) Process for the manufacture of disubstituted amines

(76) Inventors: Rudolf Schmid, Basel (CH); Rene Trussardi, Birsfelden (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/330,317

(22) Filed: Jan. 11, 2006

(30) Foreign Application Priority Data
Jan. 13, 2005 (EP) 05100180.8

(51) Int. Cl. C07K 7/06

(52) U.S. Cl. 530/330; 564/336; 548/537

(57) ABSTRACT

The present invention relates to the manufacture of the compounds of formula (I) 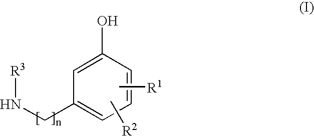 said compounds of formula (I), or their lithium salts, are valuable intermediates in the manufacture of Dolastatin 10 analogues, which are useful in the treatment of cancer.

### PRIORITY TO RELATED APPLICATIONS

This application claims the benefit of European Application No. 05100180.8, filed Jan. 13, 2005, which is hereby incorporated by reference in its entirety.

### FIELD OF THE INVENTION

The present invention relates to a new process for the manufacture of disubstituted amines. The amines obtainable by the process according to the present invention are valuable intermediates in the manufacture of Dolastatin 10 analogues.

### BACKGROUND OF THE INVENTION

Dolastatin 10 is known to be a potent antimitotic peptide, isolated from the marine mollusk Dolabella auricularia, which inhibits tubulin polymerization and is a different chemical class from taxanes and vincas (Curr. Pharm. Des. 1999, 5: 139-162). Preclinical studies of Dolastatin 10 have demonstrated activities against a variety of murine and human tumors in cell cultures and animal models. Dolastatin 10 and two synthetic dolastatin derivatives, Cemadotin and TZT-1027 are described in Drugs of the future 1999, 24(4): 404-409. Subsequently it had been found that certain Dolastatin 10 derivatives having various thio-groups at the dolaproine part show significantly improved anti-tumor activity and therapeutic index in human cancer xenograft models (WO 03/008378).

Dolastatin 10 and its derivatives consist of 5 subunits, the Dov-, Val-, Dil-, Dap- and Doe subunits.

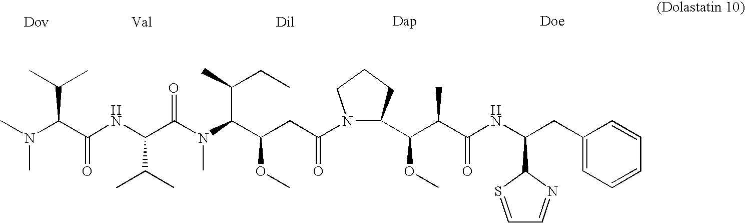

The synthesis of these compounds, including the one disclosed in WO 03/008378, is laborious and suffers from low yields, mainly due to losses over the many reaction steps required to obtain each subunit and subsequently the final product. For example, previously known synthesis routes towards the modified Doe subunit typically use a 4-step synthesis (see, e.g., H. Hashima, M. Hayashi, Y. Kamano, N. Sato, Biorg. Med. Chem, 2000, 8, 1757). Therefore, there remains a need for new and improved processes for the manufacture of Dolastatin 10, its derivatives, and each of the corresponding subunits.

### SUMMARY OF THE INVENTION

The present invention addresses this problem by providing a new, improved process for the manufacture of compounds of general formula (I) or a salt thereof wherein formula I is: 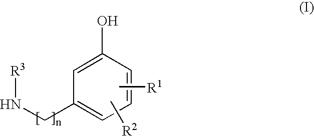
wherein R¹ and R² independently from each other are selected from the group consisting of:
   (1) halogen;
   (2) C₁-C₈-alkoxycarbonyl;
   (3) sulfamoyl;
   (4) C₁-C₈-alkylcarbonyloxy:
   (5) carbamoyloxy;
   (6) cyano;
   (7) mono- or di-C₁-C₈-alkylamino;
   (8) C₁-C₈-alkyl;
   (9) C₁-C₈-alkoxy;
   (10) phenyl;
   (11) phenoxy;
   (12) trifluoromethyl;
   (13) trifluoromethoxy;
   (14) C₁-C₈-alkylthio;
   (15) hydroxyl;
   (16) C₁-C₈-alkylcarbonylamino;
   (17) heterocyclyl;
   (18) 1,3-dioxolyl;
   (19) 1,4-dioxolyl;
   (20) amino; and
   (21) benzyl;
R³ is C₁-C₄ alkyl; and
n is 2, 3 or 4

Formula (I) represents the modified Doe subunit in the synthesis of the above-mentioned Dolastatin 10 derivatives. It has now been found that the process of the present invention provides a one-step synthesis route towards the compounds of formula (I), which is a significant improvement in the synthesis of said dolastatin 10 derivatives. In particular, the manufacture of the compounds of present invention comprises:
(a) reacting a compound of formula (II) or a salt thereof with hydroiodic acid in the presence of phosphorous or hypophosphorous acid to obtain the compounds of formula (I), wherein formula (II) is: 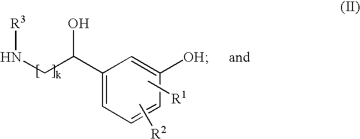
(b) optionally converting the reaction product of step (a) into the compounds of formula (III) by adding lithium hydroxide, wherein formula (III) is: 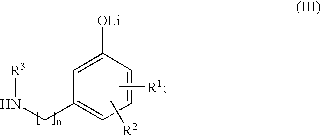
wherein R¹ and R² independently from each other are selected from the group consisting of:
   (1) halogen;
   (2) C₁-C₈-alkoxycarbonyl;
   (3) sulfamoyl;
   (4) C₁-C₈-alkylcarbonyloxy:
   (5) carbamoyloxy;
   (6) cyano;
   (7) mono- or di-C₁-C₈-alkylamino;
   (8) C₁-C₈-alkyl;
   (9) C₁-C₈-alkoxy,
   (10) phenyl;
   (11) phenoxy;
   (12) trifluoromethyl;
   (13) trifluoromethoxy;
   (14) C₁-C₈-alkylthio;
   (15) hydroxyl;
   (16) C₁-C₈-alkylcarbonylamino;
   (17) heterocyclyl;
   (18) 1,3-dioxolyl;
   (19) 1,4-dioxolyl;
   (20) amino; and
   (21) benzyl;
R³ is C₁-C₄ alkyl;
n is 2, 3 or 4; and
k is 1, 2 or 3.

In addition to providing a new and improved process for the manufacture of the compounds of formula (I), the lithium compounds of formula (III) are new and are additional embodiments of the present invention. The present invention also provides the compounds of formula (I) made by the manufacturing processes described above. In addition, the present invention provides Dolastatin 10 and its derivatives made by the manufacturing processes described herein.

### DETAILED DESCRIPTION

The term “C₁-C₄ alkyl” or “C₁-C₈ alkyl” as used herein means a straight-chain or branched-chain hydrocarbon group containing a maximum of 4 or 8 carbon atoms respectively. Examples of such alkyl groups are methyl, ethyl, n-propyl, 2-methylpropyl (iso-butyl), 1-methylethyl (iso-propyl), n-butyl, 1,1-dimethylethyl (t-butyl or tert-butyl ) or t-pentyl, and the like. The alkyl groups may be unsubstituted or may be substituted with one or more substituents, preferably with one to three substituents, most preferably with one substituent. The substituents may be selected from the group consisting of hydroxy, alkoxy, amino, mono- or di-alkylamino, acetoxy, alkylcarbonyloxy, carbamoyloxy, alkoxycarbonyl, carbamoyl, alkylcarbamoyloxy, halogen, cycloalkyl and phenyl. The C₁-C₄ alkyl group of R³ is preferably a methyl group.

The term “C₁-C₈ alkoxy” means —O—(C₁-C₈ alkyl), wherein “C₁-C₈ alkyl” has the meaning given previously.

The term “C₁-C₈alkylthio” means —S—(C₁-C₈alkyl), wherein “C₁-C₈alkyl” has the meaning given previously.

The term “cycloalkyl” as used herein means a saturated mono- or bicyclic hydrocarbon group, containing from 3 to 10 carbon atoms, preferably from 3 to 7 carbon atoms, and more preferably 5 or 6 carbon atoms. Examples of such cycloalkyls are cyclopropyl, cyclopentyl, cyclohexyl, cycloheptyl and decahydro-naphthalene.

The term “heterocyclyl” as used herein means a cycloalkyl group as defined previously, wherein 1, 2 or 3 carbon atoms (preferably 1 or 2 carbon atoms) are replaced by a N, S or O heteroatom. Examples for such heterocyclyl groups are morpholinyl, piperidinyl, piperazinyl, [1,4]oxathianyl, pyrrolidinyl, tetrahydrothiophenyl and the like.

The term “sulfamoyl” as used herein refers to the group —S(O)₂—NH₂.

The term “carbamoyl” refers to the group —C(O)—NH₂ and the term “carbamoyloxy” refers to the group —O—C(O)—NH₂.

The term “C₁-C₈-alkylcarbamoyloxy” refers to a C₁-C₈-alkyl group as defined previously attached to a parent structure via a carbamoyloxy radical, such as —O—C(O)—NH—(C₁-C₈ alkyl).

The term “C₁-C₈-alkylcarbonyloxy” refers to a C₁-C₈-alkyl group as defined previously attached to a parent structure via a carbonyloxy radical, such as alkyl-C(O)—O—. The group “C₁-C₈-alkylcarbonyloxy” therefore refers to the group C₁-C₈-alkyl-O—C(O)—.

The term “C₁-C₈-alkylcarbonylamino” refers to a C₁-C₈-alkyl group as defined previously attached to a parent structure via a carbonylamino radical, such as C₁-C₈-alkyl-C(O)—NH—.

The term “halogen” refers to fluorine, bromine, iodine or chlorine.

The term “room temperature (rt)” as used herein means the ambient temperature of the place where the process according to the present invention is carried out. Accordingly said “room temperature” can be a temperature between 15° C. and 35° C., preferably between 18° C. and 27° C., and most preferably between 18° C. and 23° C.

As used herein, the term “a therapeutically effective amount” of a compound means an amount of compound that is effective to prevent, alleviate or ameliorate symptoms of disease or prolong the survival of the subject being treated. Determination of a therapeutically effective amount is within the skill in the art.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention are contemplated. Supplementary active compounds can also be incorporated into the compositions.

The salts of compounds of formulae (I) or (II) can be obtained by conventional acid addition to said compounds; a procedure which is well known to the skilled artisan. Preferably said salts of formulae (I) or (II) are obtained by the addition of mineral acids. The term “mineral acid” is well known to one skilled in the art for representing an inorganic acid, such as hydrochloric acid, nitric acid, sulfuric acid and the like. According to the present invention the use of hydrochloric acid for the formation of said salts of formulae (I) or (II) is especially preferred.

An embodiment of the present invention, is the process for the manufacture of the compounds of formula (I) as described previously, wherein:
R³ is methyl;
n is 2; and
k is 1.

Another embodiment of the present invention is the process as described previously, wherein the compound of formula (2) or a salt thereof is reacted with hydroiodic acid in the presence of phosphorous- or hypophosphorous acid to obtain the compound of formula (1) or a salt thereof; wherein formula (1) is: 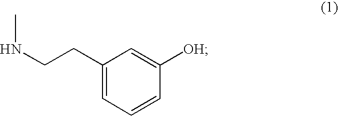
and wherein formula (2) is:

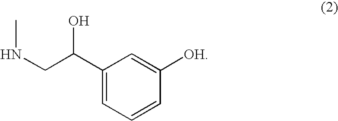

In another embodiment of the present invention, a compound of formula (2a) or a salt thereof is reacted with hydroiodic acid in the presence of phosphorous- or hypophosphorous acid to obtain the compound of formula (1) or a salt thereof; wherein formula (1) is: 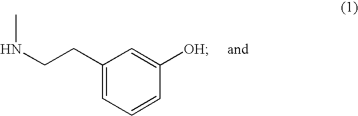
wherein formula (2a) is:

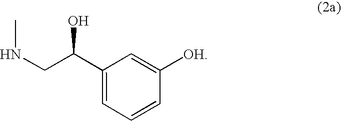

A particular embodiment of the present invention is any of the processes as described previously, wherein said reaction with hydroiodic acid is carried out in the presence of hypophosporous acid.

A particular embodiment of the present invention is any of the processes as described previously, wherein said reaction with hydroiodic acid is carried out in the presence of phosporous acid.

Still another particular embodiment of the present invention is any of the processes as described previously, wherein said reaction is carried out in the presence of 2 to 3 equivalents of hydroiodic acid.

Still another embodiment of the present invention is any of the processes as described previously, wherein said reaction is carried out in the presence of 2.5 equivalents of hydroiodic acid.

Still another embodiment of the present invention is any of the processes as described previously, wherein said reaction is carried out at temperatures between room temperature and 120° C.

Still another embodiment of the present invention is any of the processes as described previously, wherein said reaction is carried out at temperatures between 50° C. and 110° C.

The present invention also relates to a reaction of the compounds of formula (I) or salts thereof, with lithium hydroxide to obtain the respective compounds of formula (III): 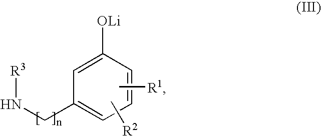
wherein R¹, R², R³ and n have the significances given previously.

In yet another embodiment of the present invention, the compound of formula (1) (as described previously) or a salt thereof is further reacted with lithium hydroxide to obtain the compound of formula (3) or a salt thereof wherein formula (3) is:

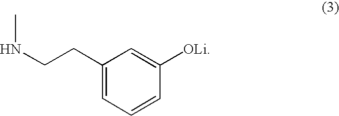

The present invention also provides the compounds of formula (III): 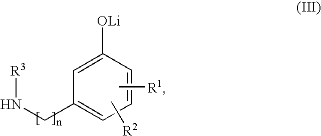
wherein R¹ and R² independently from each other are selected from the group consisting of:
   (1) halogen;
   (2) C₁-C₈-alkoxycarbonyl;
   (3) sulfamoyl;
   (4) C₁-C₈-alkylcarbonyloxy:
   (5) carbamoyloxy;
   (6) cyano;
   (7) mono- or di-C₁-C₈-alkylamino;
   (8) C₁-C₈-alkyl;
   (9) C₁-C₈-alkoxy;
   (10) phenyl;
   (11) phenoxy;
   (12) trifluoromethyl;
   (13) trifluoromethoxy,
   (14) C₁-C₈-alkylthio;
   (15) hydroxy;
   (16) C₁-C₈-alkylcarbonylamino;
   (17) heterocyclyl;
   (18) 1,3-dioxolyl;
   (19) 1,4-dioxolyl;
   (20) amino; and
   (21) benzyl;
R³ is C₁-C₄ alkyl; and
n is 2, 3 or 4.

In one particular embodiment, the present invention provides 2-(3-Hydroxyphenyl)-ethyl-methyl-amine, lithium salt.

Still another embodiment of the present invention is a process wherein the compounds of formulae (I) or a salt thereof, or formula (III) are further reacted to give the compounds of formula (A): 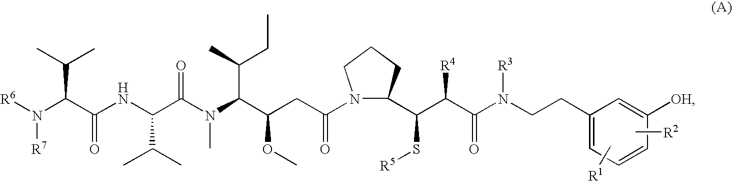
wherein said process comprises:
(a) reacting the compounds of formulae (I) or salts thereof, or the compounds of formulae (III), with an N-protected 3-pyrrolidin-2-yl-propionic acid derivative of the formula (B): 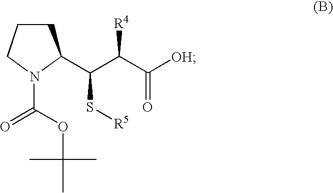
(b) chemically cleaving the tert-butoxycarbonyl group at the pyrrolidine N-atom of the reaction product of step (a) to obtain the compounds of formula (C): 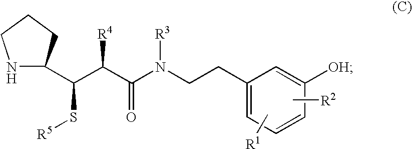
(c) further reacting the compounds of formula (C) with the compounds of formula (D): 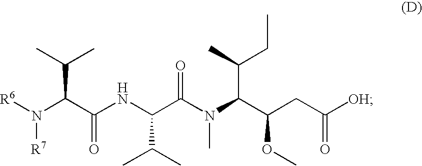
to obtain the compounds of formula (A); wherein:
R¹, R² and R³ are defined according to formula I; and
R⁴, R⁵, R⁶ and R⁷ independently from each other represent C₁-C₄-alkyl.

Still another embodiment of the present invention is the process for the manufacture of the compound of formula (A-1): 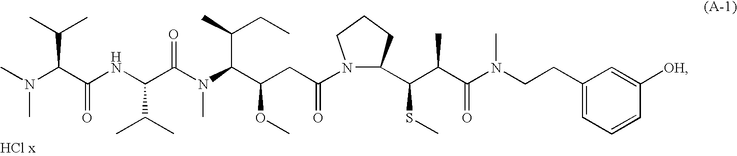
wherein said process comprises:
(a) reacting the compound of formula (1) or a salt thereof, or the compound of formula (3), with the compound of formula (B-1); 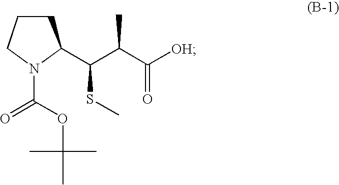
(b) cleaving the tert-butoxycarbonyl protecting group at the pyrrolidine N-atom of the reaction product of step (a) to obtain the compound of formula (C-1): 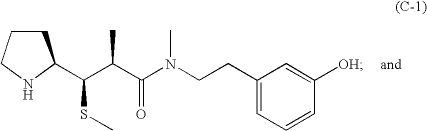
(c) further reacting the compound of formula (C- 1) with the compound of formula (D-1): 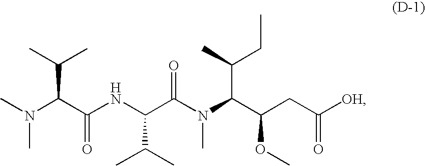
to obtain the compound of formula (A-1).

Another embodiment of the present invention is a compound of formula (A) or a salt thereof made by the process described previously for the manufacture of compounds of formula (A). Another embodiment of the present invention is a compound of formula (A-1) made by the process described previously for the manufacture of compounds of formula (A-1).

Another embodiment of the present invention is a compound of formula (I) or a salt thereof made by a process described previously for the manufacture of compounds of formula (I). Another embodiment of the present invention is a compound of formula (III). Another embodiment of the present invention is a compound of formula (III) made by a process described previously for the manufacture of compounds of formula (III). Another embodiment of the present invention is a compound of formula (1) or a salt thereof made by a process described previously for the manufacture of compounds of formula (1). Another embodiment of the present invention is a compound of formula (3) made by a process described previously for the manufacture of compounds of formula (3).

Still another embodiment of the present invention is the use of a compound of the formula (I) or a salt thereof (made by the process according to the present invention) in the manufacture of the compounds of formula (A) as defined previously.

Still another embodiment of the present invention is the use of a compound of the formula (III) as defined previously in the manufacture of the compounds of formula (A) as defined previously.

Still another embodiment of the present invention is the use of the compound of formula (1) or a salt thereof (made by the process according to the present invention) in the manufacture of the compound of formula (A-1) as defined previously.

Still another embodiment of the present invention is the use of the compound of formula (3) as defined previously in the manufacture of the compound of formula (A-1) as defined previously.

Compounds of formula (A) or (A-1) or their pharmaceutically acceptable salts made by the processes described above for the manufacture of compounds of formula (A) and (A-1) can be used as medicaments, e.g. in the form of pharmaceutical compositions. The pharmaceutical compositions can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions. Such pharmaceutical compositions may be used for the inhibition of tumor growth or for the treatment of cancer.

The above-mentioned pharmaceutical compositions can be obtained by processing the compounds of formula (A) or (A-1) or their pharmaceutically acceptable salts made by the processes described above with pharmaceutically inert, inorganic or organic carriers. For example, lactose, corn starch or derivatives thereof, talc, stearic acids or its salts and the like can be used as carriers for tablets, coated tablets, dragées and hard gelatine capsules. Suitable carriers for soft gelatine capsules are, for example, vegetable oils, waxes, fats, semi-solid and liquid polyols and the like. However, depending on the nature of the active substance, carriers may not be required for some soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They may also contain other therapeutically valuable substances.

The process of the present invention can be performed according to the following general reaction scheme (scheme 1), wherein unless explicitly otherwise stated R¹, R², R³, k and n are defined according to formula (I) recited previously. It is understood that the compounds of formulae (I) and (II) of scheme 1 also include their salts as defined previously.

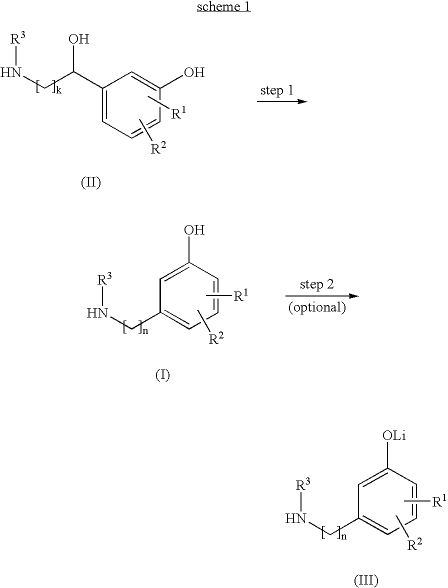

Step 1: Smooth deoxygenation is accomplished with hydroiodic acid (commercial aqueous solutions of 45-70%, preferably 55-58%) in the presence of phosphorous acid, which can be used as such or as a commercially available aqueous solution (˜50%), at reflux temperature, whereby the phosphorous acid serves to reduce the iodine formed in the reaction to iodide. The redox process is indicated by the color change of the reaction mixture from yellow at the beginning to dark brown during and to pale yellow at the end of the reaction. Aqueous hypophosphoric acid (˜50%), as for example commercially available, serves as well as phosphorous acid for reduction of the iodine formed. The phosphorous—as well as the hypophosphorous acid—can be used in amounts ranging from 0.9 to 1.5 equivalents, preferably 1.0 to 1.2 equivalents, most preferably in a slight excess of 1.1 equivalents. The hydroiodic acid can be used in catalytic amounts since it is recovered during the reaction cycle. Preferably it is used in stoichiometric amounts or in slight excess. Most preferably, hydroiodic acid serves as reactant and at the same time as the solvent for the reaction. In such cases hydroiodic acid is used in amounts of 2.0 to 3.0 equivalents, preferably in 2.5 equivalents. Due to its exothermic characteristics, the reaction is carried out at temperatures between room temperature and 120° C., preferably at temperatures between 50° C. and 110° C. The compounds of formula (I) can be isolated after neutralization of the reaction mixture with suitable bases, preferably with potassium hydroxide, extraction of the water-soluble compounds of formula (I) with 1-butanol and final distillation.

Step 2: Alternatively, in order to avoid the high-vacuum distillation, the product can be isolated as the Li salts of formula (III) by treatment of the crude product with lithium hydroxide in tetrahydrofuran. Said Li salts of formula (III) can directly be used in the further reaction sequences to obtain the respective dolastatin 10 derivatives of formulae (A) or (A-1) as defined previously.

The following examples are provided to aid the understanding of the present invention. It is understood that modifications can be made without departing from the spirit of the invention.

If not explicitly otherwise stated, the following abbreviations are used and have the following meanings:
min refers to minute(s);
h refers to hour(s);
rt refers to room temperature;
NMR refers to nuclear magnetic resonance;
GC refers to gas chromatography;
TLC refers to thin layer chromatography,
HPLC refers to high performance liquid chromatography;
The abbreviation “ca.” refers to “circa” and means “about” or “approximately”; and
mp refers to melting point.

### EXAMPLES

### Example 1

### Synthesis of 2-(3-Hydroxyphenyl)-ethyl-methyl-amine (1)

A reaction flask was charged with 50.92 g L-(−)-phenylephrine hydrochloride (2a×HCl; 250 mmol) and 82.5 ml hydriodic acid (625 mmol; 57% aqu. solution). While stirring, 22.55 g phosphorous acid (275 mmol) were added to the resulting yellow solution, whereupon the internal temperature decreased slightly. The suspension was heated in an oil bath (oil bath temperature 100° C.). At ca. 50-55° C. internal temperature the reaction started, the color of the reaction mixture turned to dark-brown and the internal temperature rose for a short time to maximally 111° C. The reaction course was monitored by HPLC analysis. The dark-brown reaction mixture was stirred at 100-105° C. for ca. 80 min resulting in a light yellow solution. This solution was cooled to 0-5° C., and 105.5 ml aqueous potassium hydroxide solution (50% aqueous solution, 13.51 M; 1.425 mol) were added dropwise in the course of 1 h while keeping the temperature at below 20° C., to attain a final pH of 11.0. The milky suspension was transferred to a separatory funnel and extracted twice with 80 ml 1-butanol. The organic phases were combined, dried over ca. 100 g sodium sulfate, filtered and the filter cake was washed with 40 ml 1-butanol. The combined filtrate and wash solution was evaporated on a rotary evaporator at 40° C./10 mbar. After distillation of ca. 100 ml of 1-butanol the remaining solution (ca. 250 ml) was transferred to a 500 ml 2-necked round bottom flask. Distillation over a Hickmann distillation apparatus afforded 23.72 g (62.7%) of the title compound as a highly viscous, colorless oil which congealed to a rigid glass at rt. b.p. 117-129° C./0.4-0.02 mbar (oil bath temp. 150-185° C.).

¹H-NMR (300 MHz, CDCl₃): 7.20 (t, J=7.8, 1 arom. H); 6.71 (d with fine structure, J=7.8, 2 arom. H); 6.65 (s with fine structure, 1 arom. H); ca. 5.9 (very br, ca. 2 H); 2.92 and 2.80 (2 t, J=6.2; 2 —CH₂—); 2.42 (s, CH₃).

### Example 2

### Synthesis of 2-(3-Hydroxyphenyl)-ethyl-methyl-amine Lithium Salt (3)

A reaction flask was charged with 330 ml hydriodic acid (2.50 mol; 57% aqu. solution) and 203.7 g L-(−)-phenylephrine hydrochloride (2a×HCl, 1.00 mol). Then, 90.20 g phosphorous acid (1.10 mol) were added to the resulting yellow solution, whereupon the internal temperature decreased to 7° C. The resulting suspension was heated in an oil bath (oil bath temperature 100° C.). After ca. 20 min, at an internal temperature of 50-55° C. the reaction started, some gas evolution occurred, the color of the reaction solution turned from yellow to black-brown, and the internal temperature rose for a short time to maximally 112° C. The progress of the reaction was monitored by HPLC. The black-brown reaction mixture was stirred at 100-105° C. for 30 min resulting in a light yellow solution. The solution was cooled to 0-5° C., and 365.0 ml potassium hydroxide (50% aqu. solution; 13.51 M; 4.93 mol) were added dropwise in the course of 1 h while maintaining a temperature range of 0-20° C., to attain a final pH of 10.1. The light yellow solution was transferred to a separatory funnel, and extracted twice with 320 ml 1-butanol. The combined light yellow organic phases were evaporated on a rotary evaporator at 40-45° C./10 mbar to obtain 253.49 g of a yellow oil containing 2-(3-Hydroxyphenyl)-ethyl-methyl-amine, 1-butanol, water and some solid potassium iodide. This mixture was treated with 1270 ml tetrahydrofuran and 253 g sodium sulfate. The suspension was stirred vigorously at rt for 1 h, then filtered over a G3 glass filter funnel, and the filter cake was washed with 400 ml tetrahydrofuran. The combined filtrate and wash solution were evaporated at 40° C./10 mbar to obtain 238.95 g of a yellow oil containing 2-(3-Hydroxyphenyl)-ethyl-methyl-amine and potassium iodide.

### Formation of the Lithium Salt

A 2 l 4-necked round bottom flask equipped with thermometer, reflux condenser, mechanical stirrer and inert gas supply was charged with the above yellow oil (238.95 g), 1200 ml tetrahydrofuran and 52.45 g lithium hydroxide monohydrate (1.25 mol). The yellow cloudy mixture was heated to reflux for 5 min, then cooled to 40-45° C. and filtered over a glass fibre filter (GF-1). The resulting clear yellow solution was cooled to 20-25° C. whereupon crystallization started. After 3 h, the white suspension was cooled to 0-5° C. and stirred at this temperature for another 18 h. The white suspension was filtered over a pre-cooled (0-5° C.) G3 glass filter funnel, the filter cake washed portionwise with pre-cooled (0-5° C.) 400 ml tetrahydrofuran and the white solid was dried in vacuo (40° C./10 mbar/12 h) to obtain 134.17 g of 2-(3-Hydroxyphenyl)-ethyl-methyl-amine Lithium Salt as white crystalline material containing 6.28% w/w of tetrahydrofuran by residual solvent analysis and 3.65% w/w of water by microanalysis. HPLC quant. assay (against internal standard) 90.0%; assay-corrected yield 76.8%.

m.p.: dec. starting from 181° C.

¹H-NMR (400 MHz, d₆-DMSO): 6.75 (t, J=7.6, 1 arom. H); 6.27 (d br, 2 arom. H); 6.0 (s br, 1 arom. H); 2.62 (m, —CH₂—); 2.46 (m, —CH₂—); 2.27 (d, J=6.0, CH₃); 1.26 (m, NH).

### Example 3

### Alternative Preparation of 2-(3-Hydroxyphenyl)-ethyl-methyl-amine Lithium Salt (3) with Hypophosporous Acid

In a 350 ml four-necked round bottom flask equipped with a thermometer, a mechanical stirrer and an inert gas supply 50.92 g L-(−)-phenylephrine hydrochloride (2a×HCl, 250 mmol) was dissolved in 83 ml hydriodic acid (57 wt % aqu. solution, 625 mmol). To the yellow solution 15 ml hypophosphorous acid (50 wt % aqu. solution, 137.5 mmol) was added. The yellow solution was heated in an oil bath (oil bath temperature 105° C.). At ca. 50-55° C. the reaction started, the reaction temperature rose to 100° C. and the color of the reaction mixture turned from yellow to black-brown. After 2 h at 95° C. the reaction mixture turned back to a yellow solution. The yellow solution was cooled to 0-5° C., and 70 ml potassium hydroxide (50 wt % aqu. solution) was added dropwise in the course of 30 min, while maintaining a temperature range of 0-20° C., to attain a final pH of 10.1. The cloudy mixture was transferred to a separatory funnel and extracted twice with 80 ml, in total with 160 ml 1-butanol. The combined light yellow organic phases were evaporated on a rotary evaporator and the residue (66.37 g of yellow oil) was dissolved in 330 ml tetrahydrofuran and treated with 13 g anhydrous sodium sulfate. The suspension was stirred at rt for 1 h, then filtered over a glass filter funnel, and the filter cake was washed with 100 ml tetrahydrofuran. The combined filtrate and wash solution were evaporated on a rotary evaporator at 40° C./400-10 mbar to obtain 62.78 g of yellow oil. The crude product was dissolved in 315 ml tetrahydrofuran and treated with 14.57 g lithium hydroxide monohydrate (347 mmol). The yellow cloudy mixture was heated to reflux for 5 min, cooled to rt within 1 h and then cooled to 0-5° C. for 18 h. The white suspension was filtered over a pre-cooled glass filter funnel and the filter cake was washed with 100 ml pre-cooled tetrahydrofuran. The white crystals were dried (40° C./10 mbar/12 h) to obtain 19.7 g of 2-(3-Hydroxyphenyl)-ethyl-methyl-amine Lithium Salt containing 2.93% w/w of water by microanalysis. HPLC quant. assay (against internal standard) 96.1%; assay-corrected yield 48%.

m.p.: dec. starting from 210° C.

Microanalysis calc. for C₉H₁₂NOLi(0.26 H₂O) (161.83): C 66.80, H 7.80, N 8.66, Li 4.29; H₂O 2.89; found: C 66.94, H 7.85, N 8.17/8.34, Li 4.12; H₂O 2.93.

### Example 4

### Synthesis of (2S)-2-((1R, 2S)-2-{[2-(3-Hydroxy-phenyl)-ethyl]-methyl-carbamoyl}-1-methylsulfanyl-propyl)-pyrrolidine-1-carboxylic acid tert-butyl ester (4)

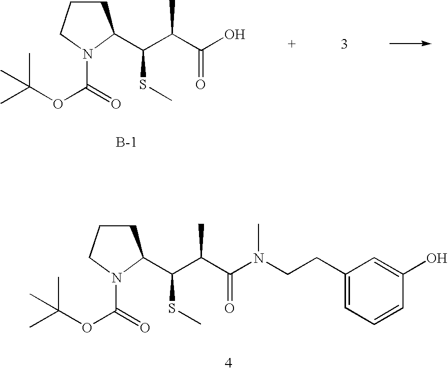

To a solution of 16.95 g 2-(3-hydroxyphenyl)-ethyl-methyl-amine lithium salt (3; 97.1 mmol) in 190 ml tetrahydrofuran 14.68 ml methanesulfonic acid were added at rt and within 2 min, whereupon the temperature rose to 61° C. The turbid, grayish solution was stirred for 5 min, then 54.07 ml triethylamine were added at rt and within 5 min, whereupon the temperature rose to 31° C. The light grey solution was stirred at rt for 10 min, then 19.64 g (2S) -2-[(1R,2S)-2-carboxy-1-methylsulfanyl-propyl]-pyrrolidine-1-carboxylic acid tert-butyl ester (B-1; 64.73 mmol) were added. To the resulting light yellow solution 12.42 g 1-hydroxy-benzotriazole hydrate (80.92 mmol) were added at rt, followed by addition of 35.78 g (benzotriazol-1-yloxy)-tris(dimethylamino)-phosphonium hexafluorophosphate (80.92 mmol), whereby the temperature rose to 39° C. The light yellow solution was stirred at rt for 60 min, whereupon HPLC indicated almost complete conversion. The yellow solution was stirred at rt for additional 1.5 h, then diluted with 85 ml tert-butyl methyl ether. The solution was washed successively with 2×190 ml hydrochloric acid (1 M) and with 2×190 ml sodium hydrogencarbonate solution (1 M), then dried over ca. 90 g sodium sulfate, filtered and evaporated (40° C./10 mbar) to provide 30.93 g of a viscous yellow oil. This material contained 78.2% of the title product 4 and 7.3% of the phenol ester by-product tert-butyl (2S)-2-[(1R,2S)-3-(3-{2-[[(2S,3R)-3-[(2S)-1-(tert-butoxycarbonyl)pyrrolidin-2-yl]-2-methyl-3-(methylthio)propanoyl]-(methyl)amino]ethyl}phenoxy)-2-methyl-1-(methylthio)-3-oxopropyl]pyrrolidine-1-carboxylte (i.e. the title compound 4 esterified at phenol with B-1) as verified by HPLC. All four aqueous wash solutions were back-extracted with 190 ml tert-butyl methyl ether and the combined extracts were dried, filtered and evaporated to give an additional 2.32 g of a viscous yellow oil. This material contained 81.0% product (of the title compound 4) but none of the phenol ester by-product as again verified by HPLC. The materials were combined to provide 32.71 g of crude product (2S)-2-((1R,2S)-2-{[2-(3-Hydroxy-phenyl)-ethyl]-methyl-carbamoyl}-1-methylsulfanyl-propyl)-pyrrolidine-1-carboxylic acid tert-butyl ester.

### Sodium Hydroxide Treatment to Saponify Phenol Ester By-Product

32.6 ml sodium hydroxide (28%; 9.1 M; 297 mmol) were added to a solution of 32.71 g of the above crude product (max. 74.9 mmol) in 163 ml methanol at rt and the solution was stirred at rt for 15 min. HPLC indicated complete cleavage of the phenol ester by-product. Subsequently methanol was removed in vacuo (20° C./10 mbar) and the remaining red solution was neutralized to pH 7 by addition of 17.16 ml acetic acid whereby an oil precipitated. Then 160 ml ethyl acetate were added to the mixture and the resulting clear two phases were separated. The organic phase was washed with 160 ml hydrochloric acid (1M) and with 2×160 ml sodium hydrogencarbonate (1M), dried over ca. 90 g sodium sulfate, filtered and evaporated in vacuo (40° C./40 mbar) to furnish 28.8 g of a light yellow foam (83.2% purity by HPLC).

### Chromatography

The above crude material (28.8 g) was dissolved in 20 ml ethyl acetate and subjected to chromatography on 864 g silica gel (Brunschwig 63-200 μm, 60 A) with ethyl acetate/heptane (2:1) as the eluent to afford 25.70 g of the title compound 4 as a light yellow foam (97.5% purity by HPLC).

### Crystallization

The above material (25.70 g) was treated with 186 ml diisopropyl ether and heated to reflux for 5 min. The resulting yellow solution was allowed to cool to rt, seeded with seed crystals, further cooled to 0-5° C. and stirred at this temperature for 19 h. The obtained white suspension was filtered over a pre-cooled (0-5° C.) glass filter funnel, and the filter cake was washed portionwise with pre-cooled 100 ml diisopropyl ether. The white crystalline material was dried (40° C./10 mbar/4 h) to afford 23.10 g of the title compound 4 (81.7% based on B-1) as white crystals (99.5% purity by HPLC).

m.p. 109-109.5° C.

¹H-NMR (400 MHz, CDCl₃): 7.2-7.1 (m, 1 arom. H); 6.85-6.45 (m, 3 arom. H and OH); 4.1-3.15 (m, 6H); 2.96 and 2.87 (2 s, N—CH₃, 2 rotamers); 2.9-2.6 (m, 3 H); 2.12 and 2.11 (2 s, S—CH₃, 2 rotamers); 2.0-1.65 (m, 4 H); 1.51 and 1.45 ( 2 s br, tBu, 2 rotamers); 1.26 (s br, —CH—CH₃).

Unless stated to the contrary, all compounds in the examples were prepared and characterized as described. All ranges recited herein encompass all combinations and subcombinations included within that range limit. All patents and publications cited herein are hereby incorporated by reference in their entirety.

1. A process for the manufacture of the compounds of formula (I) or a salt thereof wherein formula (I) is: 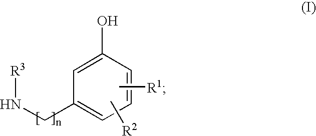
comprising reacting a compound of formula (II) or a salt thereof, wherein formula (II) is: 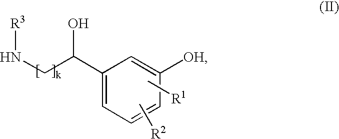
with hydroiodic acid in the presence of phosphorous acid or hypophosphorous acid to obtain a compound of formula (I) or a salt thereof;
wherein R¹ and R² independently from each other are selected from the group consisting of:
(1) halogen;
(2) C₁-C₈-alkoxycarbonyl;
(3) sulfamoyl;
(4) C₁-C₈-alkylcarbonyloxy:
(5) carbamoyloxy;
(6) cyano;
(7) mono- or di-C₁-C₈-alkylamino;
(8) C₁-C₈-alkyl;
(9) C₁-C₈-alkoxy;
(10) phenyl;
( 11) phenoxy;
(12) trifluoromethyl;
(13) trifluoromethoxy;
(14) C₁-C₈-alkylthio;
(15) hydroxyl;
(16) C₁-C₈-alkylcarbonylamino;
(17) heterocyclyl;
(18) 1,3-dioxolyl;
(19) 1,4-dioxolyl;
(20) amino; and
(21) benzyl;
R³ is C₁-C₄ alkyl;
n is 2, 3 or 4; and
k is 1, 2 or 3.

2. The process according to claim 1, wherein:
R³ is methyl;
n is 2; and
k is 1.

3. The process according to claim 1, comprising reacting a compound of formula (2) or a salt thereof, wherein formula (2) is: 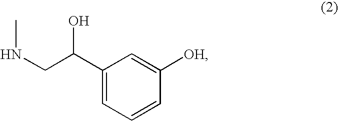
with hydroiodic acid in the presence of phosphorous acid or hypophosphorous acid to obtain a compound of formula (1) or a salt thereof, wherein formula (1) is:

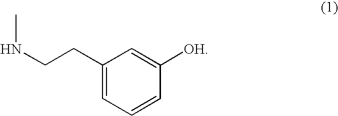

4. The process according to claim 1, comprising reacting a compound of formula (2a) or a salt thereof, wherein formula (2a) is: 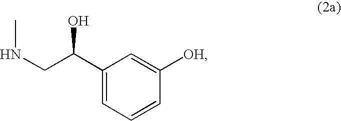
with hydroiodic acid in the presence of phosphorous acid or hypophosphorous acid to obtain a compound of formula (1) or a salt thereof, wherein formula (1) is:

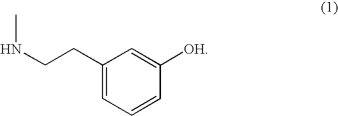

5. The process according to claim 4, wherein said reaction with hydroiodic acid is carried out in the presence of hypophosporous acid.

6. The process according to claim 4, wherein said reaction with hydroiodic acid is carried out in the presence of phosporous acid.

7. The process according to claim 1, comprising further reacting the compounds of formula (I) or a salt thereof with lithium hydroxide to obtain the compounds of formula (III): 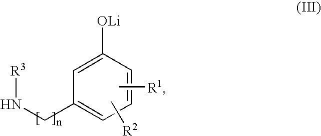
wherein R¹, R², R³ and n are defined according to claim 1.

8. A process according to claim 7, for the manufacture of the compound of formula (3): 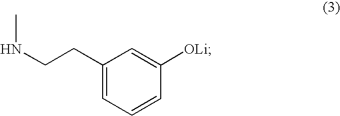
comprising reacting the compound of formula (1) or a salt thereof, wherein formula (1) is: 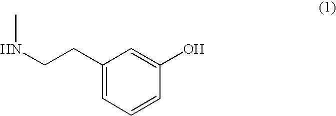
with lithium hydroxide to obtain the compound of formula (3).

9. The compounds of formula (III): 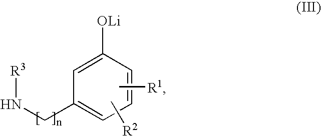
wherein R¹ and R² independently from each other are selected from the group consisting of:
(1) halogen;
(2) C₁-C₈-alkoxycarbonyl;
(3) sulfamoyl;
(4) C₁-C₈-alkylcarbonyloxy:
(5) carbamoyloxy;
(6) cyano;
(7) mono- or di-C₁-C₈-alkylamino;
(8) C₁-C₈-alkyl;
(9) C₁-C₈-alkoxy;
(10) phenyl;
( 11) phenoxy;
(12) trifluoromethyl;
(13) trifluoromethoxy,
(14) C₁-C₈-alkylthio;
(15) hydroxyl;
(16) C₁-C₈-alkylcarbonylamino;
(17) heterocyclyl;
(18) 1,3-dioxolyl;
(19) 1,4-dioxolyl;
(20) amino; and
(21) benzyl;
R³ is C₁-C₄-alkyl; and
n is 2, 3 or 4.

10. A compound according to claim 9, which is 2-(3-Hydroxyphenyl)-ethyl-methyl-amine, lithium salt.

11. The process according to claim 1, further comprising:
(a) reacting the compounds of formula (I) or a salt thereof with an N-protected 3-pyrrolidin-2-yl-propionic acid derivative of formula (B): 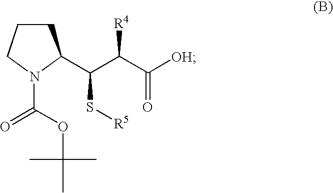
(b) cleaving the tert-butoxycarbonyl group at the pyrrolidine N-atom of the reaction product of step (a) to obtain the compounds of formula (C): 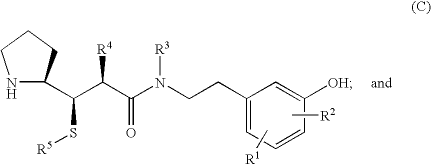
(c) further reacting the compounds of formula (C) with the compounds of formula (D): 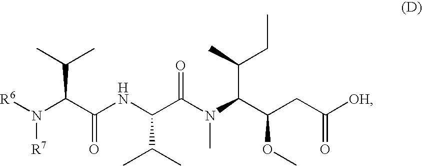
to obtain the compounds of formula (A): 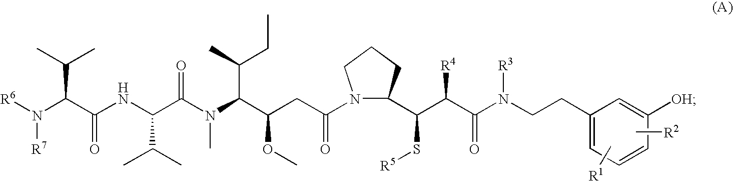
wherein:
R¹, R², and R³ are defined according to claim 1; and
R⁴, R⁵, R⁶ and R⁷ independently from each other represent C₁-C₄-alkyl.

12. The process according to claim 7, further comprising:
(a) reacting the compounds of formula (III) with an N-protected 3-pyrrolidin-2-yl-propionic acid derivative of formula (B): 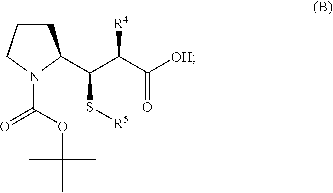
(b) cleaving the tert-butoxycarbonyl group at the pyrrolidine N-atom of the reaction product of step (a) to obtain the compounds of formula (C): 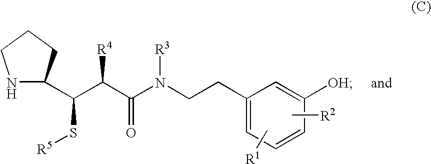
(c) further reacting the compounds of formula (C) with the compounds of formula (D): 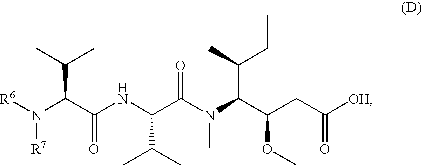
to obtain the compounds of formula (A): 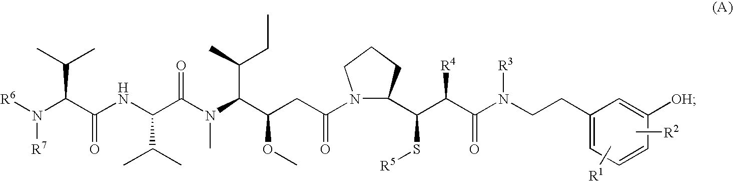
wherein:
R¹, R², and R³ are defined according to claim 1; and
R⁴, R⁵, R⁶ and R⁷ independently from each other represent C₁-C₄-alkyl.

13. A process for the manufacture of compounds of formula (A-1): 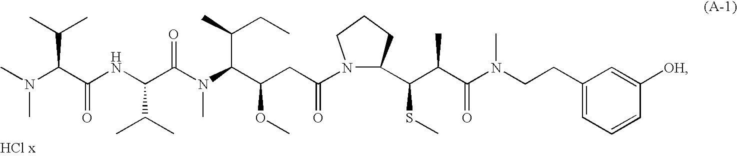
comprising:
(a) reacting the compound of formula (1) or a salt thereof as defined in claim 3 or 4 with the compound of formula (B-1): 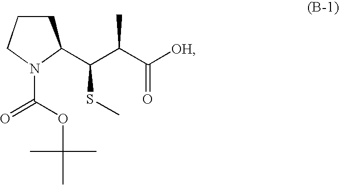
(b) cleaving the tert-butoxycarbonyl protecting group at the pyrrolidine N-atom of the reaction product of step (a), to obtain the compound of formula (C-1): 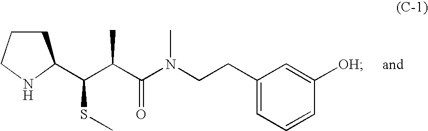
(c) further reacting the compound of formula (C-1) with the compound of formula (D-1): 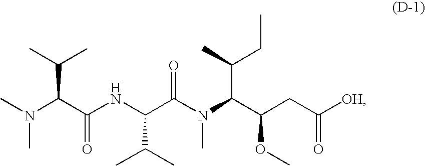
to obtain the compound of formula (A-1).

14. A process for the manufacture of compounds of formula (A-1): 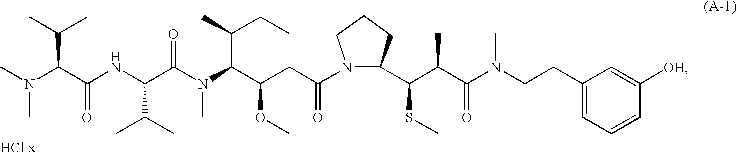
comprising:
(a) reacting the compounds of formula (3) as defined in claim 8 with the compound of formula (B-1): 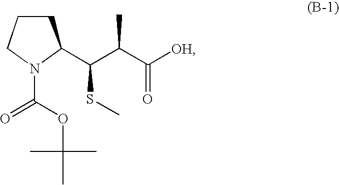
(b) cleaving the tert-butoxycarbonyl protecting group at the pyrrolidine N-atom of the reaction product of step (a), to obtain the compound of formula (C-1): 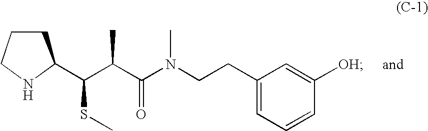
(c) further reacting the compound of formula (C-1) with the compound of formula (D-1): 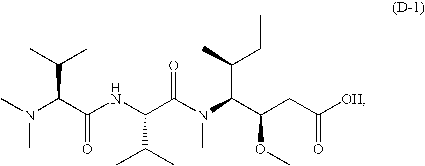
to obtain the compound of formula (A-1).

15. A compound of formula (A) or a salt thereof as defined in claim 12 made by a process according to claim 12.

16. A compound of formula (A-1) or a salt thereof as defined in claim 13 made by a process according to claim 13.

17. A compound of formula (A-1) or a salt thereof as defined in claim 14 made by a process according to claim 14.

18. A compound of formula (I) or a salt thereof as defined in claim 1 made by a process according to claim 1.

19. A compound of formula (1) or a salt thereof as defined in claim 3 made by a process according to claim 3.

20. A compound of formula (1) or a salt thereof as defined in claim 4 made by a process according to claim 4.

21. A compound of formula (3) as defined in claim 8 made by a process according to claim 8.

22. A pharmaceutical composition comprising a therapeutically effective amount of a compound of claim 15 and a pharmaceutically acceptable carrier.

23. A pharmaceutical composition comprising a therapeutically effective amount of a compound of claim 16 and a pharmaceutically acceptable carrier.

24. A pharmaceutical composition comprising a therapeutically effective amount of a compound of claim 17 and a pharmaceutically acceptable carrier.
